(19) United States
(12) Patent Application Publication
(10) Pub. No.: US 2014/0357923 A1
(43) Pub. Date: Dec. 4, 2014

(54) Vapor-Liquid Separation

(71) Applicant: ExxonMobil Chemical Patents Inc., Baytown, TX (US)

(72) Inventors: Thomas W. Pavia, Houston, TX (US); Berne K. Stober, Esmont, VA (US); Terri S. Vander Pol, Baytown, TX (US)

(21) Appl. No.: 14/178,461

(22) Filed: Feb. 12, 2014

Related U.S. Application Data
(60) Provisional application No. 61/827,918, filed on May 28, 2013.

(30) Foreign Application Priority Data
Jul. 4, 2013 (EP) 13175131.5

(51) Int. Cl. C07C 4/04; B01D 3/22

(52) U.S. Cl. CPC C07C 4/04; B01D 3/22
USPC 585/648; 202/158

(57) ABSTRACT

The invention generally relates to vapor-liquid separation, such as disengaging liquid from vapor containing C₂₋₄ olefin. The invention also relates to processes for separating liquid and vapor, to equipment useful in such processes, and to the use of such equipment in vapor-liquid separation systems.

### PRIORITY

This application claims priority to U.S. Provisional Application Ser. No. 61/827,918 filed May 28, 2013 and European Patent Application 13175131.5 filed Jul. 4, 2013, the disclosures of which are fully incorporated herein by reference.

### FIELD OF INVENTION

The invention generally relates to vapor-liquid separation, such as disengaging liquid from vapor containing C₂₋₄ olefin. The invention also relates to separation processes, to equipment useful in such processes, and to the use of such equipment in vapor-liquid separation systems.

### BACKGROUND

Many useful products are produced from olefins having a number of carbon atoms in the range of 2 to 4 (“C₂₋₄ olefins”). For example, ethylene and/or propylene can be polymerized to produce polymer, such as polyethylene, polypropylene, ethylene-propylene copolymer, etc. C₂₋₄ olefins can be produced in conventional process; e.g., by one or more of catalytically converting alcohol, such as methanol; pyrolysing a hydrocarbon-containing feed, as in steam cracking; or catalytically cracking a hydrocarbon feed, as in fluidized catalytic cracking, hydrocracking, etc.

Besides C₂₋₄ olefins, effluents from these processes generally contain molecules boiling in the naphtha boiling-range, e.g., a mixture of molecules (including C₅+ olefins) having an initial atmospheric boiling point in the range of about 25° F. (−3.9° C.) to about 35° F. (1.7° C.) and a final atmospheric boiling point in the range of about 430° F. (221° C.) to about 550° F. (288° C.). The effluent also generally contains molecules boiling in the gas oil boiling range, e.g., a mixture of molecules having an initial atmospheric boiling point that is greater than those of the naphtha boiling range and a final atmospheric of about 1050° F. (566° C.). Depending on the choice of feed, the effluent might also contain tar having an atmospheric boiling point >about 1050° F. (566° C.). When steam cracking is utilized, the effluent also generally contains water and acidic gases such as H₂S and/or CO₂. It is conventional to cool the effluent, e.g., by contacting the effluent with a quench fluid. When the effluent contains tar, it is conventional to remove at least a portion of the effluent's tar in one or more separator drums, e.g., one or more tar knock-out drums. Following effluent cooling, the gas oil fraction condenses into a liquid phase, with the naphtha boiling-range molecules and C₂₋₄ olefin remaining in the vapor phase.

A portion of the liquid phase is generally disengaged from the vapor phase in, e.g., one or more fractionators, e.g., in a primary fractionator. A vapor-phase mixture comprising (i) C₂₋₄ olefins and (ii) at least a portion of the effluent's naphtha boiling-range molecules, is separated from the effluent in the fractionator, and is conducted away for a second separation, e.g., separation of at least a portion of the mixture's naphtha boiling-range molecules and at least a portion of the mixture's C₂₋₄ olefins. Generally, the second separation is carried out in one or more flash drums, with the naphtha boiling-range molecules being conducted away from the flash drum as a liquid phase (e.g., as flash drum bottoms) and the C₂₋₄ olefins being conducted away from the flash drum as a vapor-phase (e.g., as flash-drum vapor). It is conventional to divide (e.g., split) the naphtha boiling-range molecules derived from the flash drum into two streams: a first stream which is recycled to the fractionator, e.g., as re-flux, and a second stream which can be conducted away for further processing, such as hydroprocessing and/or motor gasoline blending. The vapor phase conducted away from the flash drum can be compressed and then conducted to one or more separation stages for removal of one or more of the desired C₂₋₄ olefins. These can be stored and/or subjected to further processing, such as polymerization.

It is conventional to divide, e.g., split, the gas oil into two or more portions. The first portion of the gas oil can be returned to the primary fractionator, via a pump-around loop for example, for transferring any entrained vapors in the first portion to the primary fractionator's upper section and for maintaining the appropriate amount of liquid in the primary fractionator's lower section. The second portion of the gas oil can be utilized, e.g., for one or more of (i) quenching the effluent upstream of the primary fractionator, (ii) blending to produce fuel oil, (iii) further processing, or (iv) storage.

In response to increased demand for C₂₋₄ olefins, it is advantageous for operators to efficiently increase their capacity for producing these molecules. An operator might do this, for example, by increasing one or more of the size, capacity, yield, or number of steam cracking furnaces, in order to increase the amount of effluent available for fractionation. It is therefore desired to increase fractionation capacity, e.g., the ability of the primary fractionator to efficiently separate liquid from the effluent, while minimizing (i) any increase in the naphtha fraction's final boiling point and/or (ii) any decrease in the amount of gas oil disengaged from the effluent. It would be particularly desirable if this could be accomplished with little or no increase in the primary fractionator's internal volume.

### SUMMARY OF THE INVENTION

In certain embodiments, the invention relates to a process for separating a vapor comprising naphtha and C₂₋₄ olefins from a process stream comprising quench fluid, naphtha, and C₂₋₄ olefins, and optionally higher-boiling hydrocarbons, acidic molecules, water, etc., wherein the separated naphtha has a first final boiling point T₁. The separation can be carried out in a fractionator having (A) an elongated vessel, the vessel having an initial internal volume V₁≧1,000 ft³ (28.3 m³), e.g., ≧5,000 ft³ (142 m³), such as in the range of 7,500 ft³ (212 m³) to 15,000 ft³ (425 m³); (B) a feed inlet for conveying the process stream into the vessel; (C) an outlet for conveying the vapor away from the vessel; (D) a group of perforated fractionator trays, e.g., at least three trays, such as at least six trays, for disengaging from the process stream at least a portion of the process stream's naphtha and C₂₋₄ olefins, each of the trays being separated from its nearest neighbor trays by a first inter-tray spacing <910 mm, e.g., in the range of 300 mm 600 mm, and (E) a first vapor handling capacity C_{f1}. Certain aspects of the invention relate to an improvement which comprises:

decreasing the number in the tray group to 3, 4, or 5, wherein (i) each of the group's trays has an open area ≧0.25, (ii) each of the group's trays is separated from its nearest neighbor tray by a second inter-tray spacing in the range of from about 36 inches (910 mm) to about 50 inches (1270 mm), (iii) the fractionator has an increased vapor handling capacity C_{f2}, C_{f2} being ≧[C_{f1+}(0.01*C_{f1})], (iv) the fractionator has a final internal volume V₂, |V₂−V₁| being ≦0.1*V₁, and (v) the separated naphtha has a second final boiling point T₂, T₂−T₁ being ≦15.0° C.

In other aspects, the invention relates to a fractionator, comprising:

(a) an elongated fractionator vessel having an internal volume ≧about 28.3 m³ and a C_{f}≧0.33;
(b) a first group of three, four, or five perforated trays in the fractionator, each tray of the first group being (i) a dual-flow tray having an open area in the range of 0.15 to 0.30 and (ii) adapted to substantially prevent fluid flow between the tray's perimeter and the fractionator vessel's inner surface;
(c) a second group of three, four, or five perforated trays in the fractionator, the second group being spaced apart from the first group along the fractionator vessel's long axis, each tray of the second group being (i) a dual-flow tray having an open area ≧0.25 and (ii) adapted to substantially prevent fluid flow between the tray's perimeter and the fractionator vessel's inner surface;
(d) a first conduit for conducting to the fractionator vessel a first mixture comprising naphtha, quench fluid, and ≧10.0 wt. % C₂₋₄ olefin based on the weight of the first mixture;
(e) a second conduit for conducting away a second mixture from the fractionator vessel, the second mixture comprising naphtha and C₂₋₄ olefin, the second mixture being separated from the first mixture in the fractionator vessel, the naphtha having a final boiling point ≦288° C.; and
(f) a third conduit for conducting away from the fractionator vessel a gas oil that is separated from the first mixture in the fractionator vessel, the gas oil having an initial boiling point that is greater than or equal to the naphtha's final boiling point.

In further aspects, the invention relates to a vapor-liquid separation system, comprising:

(a) first and second vessels, the first vessel being elongated and having an internal volume ≧about 28.3 m³ and a C_{f}≧0.101 m/s;
(b) a first region of the first vessel, the first region having three, four, or five perforated trays, each tray being (i) a dual-flow tray that is spaced apart from its nearest-neighbor tray(s) along the first vessel's long axis and (ii) positioned to substantially prevent fluid flow between the tray's perimeter and the first vessel's internal surface, wherein each tray has an open area in the range of about 0.15 to about 0.30 and the tray's perforations have a size in the range of from about 13 mm to about 25 mm;
(c) a second region of the first vessel, the second region being substantially non-overlapping with the first region and having three, four, or five perforated trays, each tray being (i) a dual-flow tray that is spaced apart from its nearest-neighbor tray(s) along the first vessel's long axis and (ii) positioned to substantially prevent fluid flow between the tray's perimeter and the first vessel's internal surface, wherein each tray has an open area ≧0.25 and the tray's perforations have a size in the range of from about 13 mm to 25 mm;
(d) a first conduit for transferring to the first vessel's first region a first mixture comprising quench fluid, naphtha, and ≧10.0 wt. % C₂₋₄ olefin based on the weight of the first mixture;
(e) a second conduit for transferring a second mixture from the first vessel's second region to the second vessel, the second mixture comprising C₂₋₄ olefin that is separated from the first mixture in the first vessel;
(f) a third conduit for conducting away from the first vessel's first region a gas oil that is separated from the first mixture in the first vessel, the gas oil having an initial boiling point ≧about 221° C.;
(g) a fourth conduit for conducting a liquid naphtha away from the second vessel, wherein the liquid naphtha (i) is disengaged from the second mixture in the second vessel and (ii) has a final boiling point ≦288° C.;
(h) a fifth conduit connected to the fourth conduit for transferring a first portion of the liquid naphtha from the fourth conduit to the to the second region of the first vessel;
(i) a sixth conduit connected to the fourth conduit for conducting away from the fourth conduit a second portion of the liquid naphtha; and
(j) a seventh conduit connected to the second vessel for conducting away a vapor from the second vessel, wherein the vapor (i) comprises C₂₋₄ olefin and (ii) is disengaged from the second mixture in the second vessel.

In further aspects, the invention relates to a hydrocarbon conversion process, comprising:

(a) providing a feedstream, the feedstream comprising ≧75.0 wt. % hydrocarbon based on the weight of the feedstream;
(b) exposing the feedstream to a temperature ≧400° C. under pyrolysis conditions to produce an effluent comprising ≧5.0 wt. % of C₂₋₄ olefin based on the weight of the feedstream, wherein the pyrolysis is conducted at a feedstream mass flow rate ≧113×10³ kg per hour and a feedstream:water mass ratio in the range of from about 2.0 to 2.5;
(c) combining the effluent with a quench fluid to produce a first mixture;
(d) conducting the first mixture to a first separation zone having a C_{f}≧0.101 m/s, the first separation zone comprising substantially non-overlapping first and second regions, wherein (i) the first region contains three, four, or five dual-flow trays and (ii) the second region contains three, four, or five dual-flow trays;
(e) separating a second mixture from the first mixture in the first separation zone, and transferring the second mixture from the first separation zone to a second separation zone, the second mixture comprising C₂₋₄ olefin that is separated from the first mixture in the first separation zone;
(f) separating a gas oil from the first mixture in the first separation zone, the gas oil having an initial boiling point ≧about 221° C.; and
(g) separating a liquid naphtha from the second mixture in the second separation zone, the liquid naphtha having a final boiling point ≦288° C.

### BRIEF DESCRIPTION OF THE DRAWINGS

FIG. 1 schematically illustrates a representative process for producing C₂₋₄ olefins by steam cracking.

FIG. 2 schematically illustrates a conventional primary fractionator.

FIG. 3 schematically illustrates a primary fractionator configured according to the invention.

### DETAILED DESCRIPTION OF THE INVENTION

In many processes which utilize hydrocarbon pyrolysis for producing C₂₋₄ olefins, the pyrolysis effluent is quenched to produce a process stream (a first mixture), the process stream containing, e.g., molecular hydrogen and hydrocarbon. The quench fluid, naphtha, and the C₂₋₄ olefins are generally separated from the process stream in one or more fractionators for separating from the process stream a gas oil fraction (generally in the liquid phase) and a vapor phase (a second mixture) comprising naphtha and the C₂₋₄ olefins. For example, certain conventional processes utilize a fractionator having (i) an internal volume ≧1,000 ft³ (28.3 m³), e.g., ≧5,000 ft³ (142 m³), such as in the range of 7,500 ft³ (212 m³) to 15,000 ft³ (425 m³); (ii) a first group of fractionator trays, each of the trays being located in a lower region of the fractionator, (iii) a second group of at least six perforated dual-flow fractionator trays, each of the trays being located in an upper region of the fractionator and having an inter-tray spacing <910 mm, e.g., in the range of 300 mm 600 mm, and (iv) a vapor handling capacity (“C_{f}”)<0.30 ft/second (0.091 m/s), e.g., C_{f}≦0.26 ft/second (0.079 m/s), such C_{f}≦0.22 ft/second (0.067 m/s). Cooling and/or depressurization of the vapor, e.g., in one or more heat exchanges and/or flash drums, can be utilized for separating a liquid naphtha from the vapor to produce an upgraded vapor having an increased concentration of the desired C₂₋₄ olefin. The separated naphtha is a valuable byproduct. For example, a first portion of the separated naphtha can be returned to the fractionator (e.g., as a reflux) and a second portion of the separated naphtha can be conducted away, e.g., for hydroprocessing and/or blending to produce fuel. In certain embodiments, at least a portion of the gas oil is utilized as a quench fluid for the quenching of the pyrolysis effluent.

It is observed that increasing the fractionator's C_{f} to a value ≧0.33 ft/second (0.101 m/s), e.g. ≧0.35 ft/second (0.107 m/s), such as ≧0.37 ft/second (0.112 m/s), results in entrainment of gas oil in the vapor conducted to the flash drum, leading to a discoloration of the naphtha separated from the vapor (e.g., the separated naphtha becomes black in color), and a significant increase in the naphtha's final boiling point. This downgrading of the separated naphtha undesirably impacts its usefulness as reflux and as a fuel blendstock. Moreover, the entrainment of gas oil in the fractionator's overhead vapor undesirably lessens the amount of liquid gas oil which can be separated in the fractionator, e.g., for use as a quench fluid.

In certain embodiments, the invention is based on the discovery that this difficulty can be overcome, with little or no increase in the fractionator's internal volume and/or little or no change in the relative amount of feed/diluent subjected to the pyrolysis, by reducing the number of trays of the second group to, e.g., 3, 4, or 5 trays, increasing inter-tray spacing to a value in the range of about 36 inches (910 mm) to about 50 inches (1270 mm), with the open area of each of the trays of the second group being ≧0.25. Should the original trays of the second group have an open area <0.25, these should be replaced with dual-flow trays having an open area ≧0.25. It is observed that re-configuring the primary fractionator this way permits an increase in the fractionator's C_{f} from an initial value C_{f1} (before the reconfiguration) to a final value C_{f2} (after the reconfiguration), wherein (i) C_{f2} is ≧[C_{f1}+(zC_{f1})], where z≧0.01, e.g., z is in the range of 0.01 to 0.10, such as z=0.01, or 0.02, or 0.03, or 0.04, or 0.05; (ii) the fractionator has a final internal volume V₂ (after the reconfiguration) that is substantially the same as the fractionator's internal volume before the reconfiguration (V₁), e.g., |V₂−V₁| being ≦0.1*V₁, such as ≦0.01*V₁, and (iii) the separated naphtha has a second final boiling point T₂ (after the reconfiguration) that is substantially the same as the separated naphtha's final boiling before the reconfiguration (T₁), e.g., T₂−T₁ being ≦15.0° C., such as ≦10.0° C. Optionally, C_{f2} is ≧0.33 feet/second (0.101 m/s), e.g. ≧0.35 feet/second (0.107 m/s), such as ≧0.37 feet/second (0.112 m/s), with little or no entrainment of gas oil into the fractionator's overhead vapor. The lessened amount of gas oil entrainment is evidenced by (i) little or no discoloration of the separated liquid naphtha (the naphtha has a light tan color, and has a luminance ≧5%) and (ii) little or no increase in the separated liquid naphtha's final boiling point. This result is surprising because, although the positive influence of increased tray spacing and increased open area on overhead volumetric flow rate is known, doing so was also expected to lead to an even greater amount of naphtha discoloration and an even greater increase in naphtha final boiling point.

The invention will now be described in terms of a representative steam cracking process. The invention is not limited to this embodiment, and this description is not meant to foreclose other embodiments within the broader scope of the invention, for example those process where the process stream is derived from one or more of alcohol conversion, fixed bed catalytic cracking, fluidized catalytic cracking, coking, etc.

For all purposes of this description and appended claims, the following terms are defined as follows:

Initial boiling point of a mixture means the initial boiling point of the mixture at atmospheric pressure (also called the initial atmospheric boiling point). The initial boiling point of a mixture at atmospheric pressure is determined in accordance with ASTM Standard D86.

Final boiling point of a mixture means the final boiling point of the mixture at atmospheric pressure (also called the final atmospheric boiling point). The final boiling point of a mixture at atmospheric pressure is determined in accordance with ASTM Standard D86.

Naphtha means a mixture comprising ≧75.0 wt. % of molecules containing hydrogen and carbon, with not less than 10.0 wt. % of which mixture having an atmospheric boiling point <175° C. and not less than 95.0 wt. % of which mixture having an atmospheric boiling point ≦260° C., wherein the boiling ranges are measured in accordance with ASTM Standard D86 and the weight percents are based on the weight of the mixture.

Gas oil means a liquid, hydrocarbon-containing distillate, wherein more than 5.0 wt. % of the distillate has an atmospheric boiling point >260° C., the boiling range being measured in accordance with ASTM Standard D86, and the weight percent being based on the weight of the distillate.

Tray means a substantially horizontal, fluid-impermeable barrier located within a fractionator, the barrier being adapted to substantially prevent fluid flow between the barrier's perimeter and the fractionator vessel's internal wall, the barrier having at least one through-aperture (e.g., perforation or hole) adapted for disengaging liquid from vapor. The term tray includes those having at least one downcomer, downcomers being an opening in the tray adapted for conducting separated liquid in a flow direction countercurrent to vapor flow.

Sieve tray means a perforated plate utilized as fractionator tray, the tray optionally including one or more downcomers.

Dual-flow tray means a substantially flat sieve tray having no downcomers.

Ripple tray™ means a corrugated sieve tray having no downcomers, such as those described in U.S. Pat. No. 2,767,967. Ripple trays™ are available from Stone and Webster Process Technology, Houston, Tex.

For a sieve tray, the term “open area” means the ratio of hole area to bubbling area, where “bubbling area” means the total internal cross sectional area of the fractionator at the tray location minus the total non-perforated area of the tray, and “hole area” means the total area of tray perforation.

The term “pitch:diameter ratio” for a sieve tray whose perforations (i) have a substantially constant diameter and (ii) are arranged in a periodic array, means the minimum center-to-center distance between nearest-neighbor perforations divided by the diameter of the perforations.

It is conventional to use a support ring (or ring segment) for supporting a sieve tray within a fractionator, with a portion of the bottom of the tray located proximate to the tray's perimeter being in contact with the top surface of the support ring. With respect to a first sieve tray that is one of a plurality (e.g., group) of sieve trays located within a fractionator, the term “inter-tray spacing” means the distance between (i) the top surface of the first tray's support ring and (ii) the top surface of the support ring that is supporting the first tray's nearest-neighbor tray, the distance being measured in a direction substantially parallel to the fractionator's longest axis.

Overhead volumetric flow rate means the total mass flow rate of vapor conducted away from the fractionator region located above the uppermost tray, the mass flow rate being determined at the actual temperature of the vapor being conducted away. Overhead volumetric flow rate is generally expressed in units of actual volume per hour, where “actual volume” means the vapor's volume at 250° F. (121° C.).

A fractionator's vapor handling capacity (“C_{f}”) means (the overhead volumetric flow rate divided by the fractionator's cross sectional area) times the square root of [(the density of the vapor conducted away from the fractionator) divided by (the density of the gas oil conducted away from fractionator−the density of the vapor conducted away from the fractionator)]; wherein (i) the fractionator's cross sectional area is the numerical average of the fractionator's cross sectional area determined at the locations of the three uppermost trays and (ii) the densities are determined at the temperatures at which these streams are conducted away.

A naphtha is black in color when the naphtha has a luminance (“L”) <5.0 as determined by the CIECAM02 standard established by the Commission Internationale de l'eclairage.

A representative steam cracking process utilizes a feed which comprises ≧50.0 wt. % hydrocarbon, based on the weight of the feed. For example, in certain embodiments, the feed comprises ≧50.0 wt. %, e.g., ≧75.0 wt. %, such as ≧90.0 wt. % of naphtha and/or alkanes (including isoalkanes, cycloalkanes, etc.) having a number of carbon atoms in the range 2 to 5, e.g., from 2 to 4. In other embodiments, the feed comprises of one or more of gas oil, vacuum gas oil, crude oil, resid, or resid admixtures; including those comprising ≧about 0.1 wt. % asphaltenes. Suitable crude oils include, e.g., high-sulfur virgin crude oils, such as those rich in polycyclic aromatics. In a particular embodiment, the feed comprises about 20.0 wt. % to 80.0 wt. % of ethane and about 80.0% to about 20.0% of propane, the weight percents being based on the weight of the feed.

In certain embodiments, the amount of feed is ≧about 250×10³ pounds per hour (113×10³ kg per hour) in the range of from about 250×10³ pounds per hour (113×10³ kg per hour) to about 500×10³ pounds per hour (226×10³ kg per hour). In other embodiments, it is desired to increase the feed amount from an initial value that is <250×10³ pounds per hour (113×10³ kg per hour), e.g., in the range of about 100×10³ pounds per hour (45×10³ kg per hour to about 250×10³ pounds per hour (113×10³ kg per hour), to a final value ≧, e.g., in the range of about 250×10³ pounds per hour (113×10³ kg per hour) to about 350×10³ pounds per hour (160×10³ kg per hour).

The feed is generally combined with diluent before pyrolysis, the diluent comprising, e.g., ≧95.0 wt. % water based on the weight of the diluent. In certain embodiment, the feed and diluent are combined to provide an amount of diluent that ≧1.0 wt. % diluent based on the weight of the diluent-feed mixture, such as ≧25.0 wt. %. When the pyrolysis is steam cracking, the diluent-feed mixture can be produced by combining the feed with steam, e.g., at a ratio of 0.2 to 4.0 kg steam per kg hydrocarbon in the feed. In a particular embodiment, the feed comprises ≧95.0 wt. % hydrocarbon, and the amount of steam is in the range of about 0.4 kg of steam per kg of feed to 0.6 kg of steam per kg of feed. In certain embodiments, the amounts of feed and diluent are increased to increase the amount of process stream, but the relative amount of steam (kg of steam divided by kg of feed) is substantially constant, e.g., the final value of (kg of steam divided by kg of feed) is within +/−5% of the initial value, e.g., within about +/−1% of the initial value of (kg of steam divided by kg of feed).

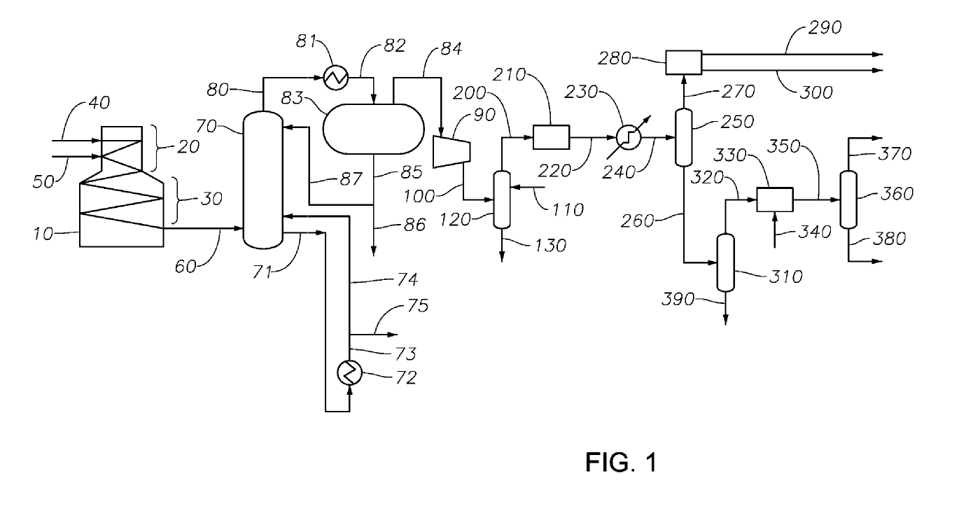

In certain embodiments shown schematically in FIG. 1, the feed is conducted via line 40 to a steam cracker furnace 10 having two main sections: a convection section 20 and a radiant section 30. The feed's hydrocarbon component enters the convection section of the furnace where it is heated and vaporized, e.g., by indirect contact with hot flue gas from the radiant section and by direct contact with the diluent, which is added to the feed via line 50 to produce the feed-diluent mixture.

Optionally, the steam cracker furnace is integrated with at least one vapor/liquid separation device (conventionally referred to as flash pot or flash drum), for upgrading the steam cracker feed or the feed-diluent mixture. Such vapor/liquid separator devices are particularly suitable when the feed comprises ≧about 0.1 wt. % asphaltenes based on the weight of the feed hydrocarbon, e.g., ≧about 5.0 wt. %. Conventional vapor/liquid separation devices can be utilized to do this, though the invention is not limited thereto. Examples of such conventional vapor/liquid separation devices include those disclosed in U.S. Pat. Nos. 7,138,047; 7,090,765; 7,097,758; 7,820,035; 7,311,746; 7,220,887; 7,244,871; 7,247,765; 7,351,872; 7,297,833; 7,488,459; 7,312,371; and 7,235,705, which are incorporated by reference herein in their entirety. Suitable vapor/liquid separation devices are also disclosed in U.S. Pat. Nos. 6,632,351 and 7,578,929, which are incorporated by reference herein in their entirety. Generally, when using a vapor/liquid separation device, the composition of the vapor phase leaving the device is substantially the same as the composition of the vapor phase entering the device, and likewise the composition of the liquid phase leaving the flash drum is substantially the same as the composition of the liquid phase entering the device, i.e., the separation in the vapor/liquid separation device consists essentially of a physical separation of the two phases entering the drum.

The feed-diluent mixture is conducted from convection section 20 to the radiant section 30, where at least a portion of the feed's hydrocarbon is exposed to a temperature >400° C. in order to convert at least a portion of the mixture's hydrocarbon molecules by pyrolysis. Optionally, the feed-diluent mixture is conducted to the radiant section via crossover piping (not shown). Suitable pyrolysis conditions in the radiant section include, e.g., exposing the feed-diluent mixture to a temperature (measured at the radiant outlet) >400° C., e.g., in the range of 400° C. to 900° C., and a pressure >0.1 bar (absolute), for a residence time in the range of from about 0.01 seconds to 5.0 seconds. For example, the steam cracking conditions can include one or more of (i) a temperature ≧760° C. (e.g., in the range of 760° C. to 880° C.); (ii) a pressure >0.5 bar (absolute (e.g., in the range of from 1.0 to 5.0 bar (absolute)), or (iii) a residence time in the range of from 0.10 to 2.0 seconds.

The radiant section's effluent (the pyrolysis effluent) is conducted away via line 60 (a first conduit), and generally comprises, e.g., gas oil, naphtha, the C₂₋₄ olefins, molecular hydrogen, acetylene, acidic gases such as H₂S and/or CO₂, mercaptans, and unconverted components of the feed-diluent mixture, such as unconverted steam. The pyrolysis effluent is generally quenched, e.g., by combining the pyrolysis effluent with quench fluid to produce a process stream having a temperature less than that of the pyrolysis effluent, the process stream comprising a liquid phase and a vapor phase. The quench fluid can be, e.g., gas oil obtained from separation stage 70.

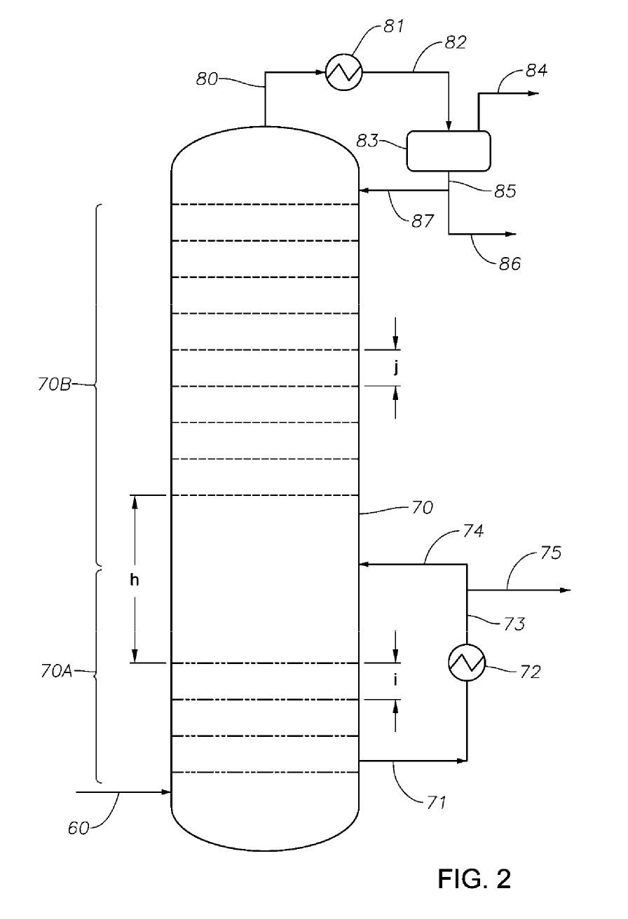

In the embodiments illustrated schematically in FIG. 1, the process stream is conducted to stage 70 via conduit 60 (a first conduit). Separation stage 70 can be, e.g., a fractionator, such as a primary fractionator. The fractionator can comprise, e.g., an elongated vessel, the vessel's long axis being oriented substantially perpendicular to the surface of the earth. A vapor stream comprising naphtha and C₂₋₄ olefin is conducted away from stage 70 via line 80 (a second conduit). Heat can be removed from the vapor, e.g., in one or more heat exchangers 81, with the vapor then conducted to a second separation stage 83 via line 82 for separating C₂₋₄ olefin and naphtha. Stage 83 can comprise, e.g., one or more vapor-liquid separators, such as one or more flash drums. An upgraded process stream comprising C₂₋₄ olefin is conducted away from stage 83 via line 84 (a seventh conduit). Separated naphtha is conducted away from stage 83 via line 85. The separated naphtha can be divided (e.g., split) into at least first and second portions, with the first portion returned to stage 70 (e.g., as reflux) via line 87 (a fifth conduit) and the second portion conducted away via line 86 (a sixth conduit), e.g., for storage or further processing (such as hydroprocessing and/or fuel blending). A gas oil fraction is conducted away from stage 70 via line 71. Line 71 (a third conduit) is generally positioned downstream of at least one tray of the first group, with “downstream” being with respect to the flow of the process stream. Heat can be removed from the separated gas oil, e.g., in heat exchanger 72, with the cooled gas oil conducted away via line 73. The cooled gas oil can be divided into at least two streams, as shown in FIGS. 1 and 2. For example, the cooled gas oil can be divided to produce (i) a first portion, which can be utilized as at least a portion of the quench fluid, e.g., by combining cooled gas oil (obtained, e.g., from line 75) with pyrolysis effluent to produce the process stream, (ii) a second portion, which can be conducted away from the process, for storage and/or further processing (such as for hydroprocessing and/or fuel oil blending), and/or (iii) a third portion, which can be returned to stage 70 via line 74, e.g., as bottoms pump-around (pump not shown). Stage 70 can utilize e.g., a fractionator vessel having an internal volume ≧1000 ft³ (28.3 m³), such as a substantially cylindrical vessel having inside diameter ≧10 feet (3.05 m), e.g., in the range of about 10 feet (3.05 m) to about 30 feet (9.15 m); and a length (e.g., height when the fractionator's long axis is substantially vertical) ≧10 feet (3.05 m), e.g., in the range of about 40 feet (12.20 m) to about 120 feet (36.6 m).

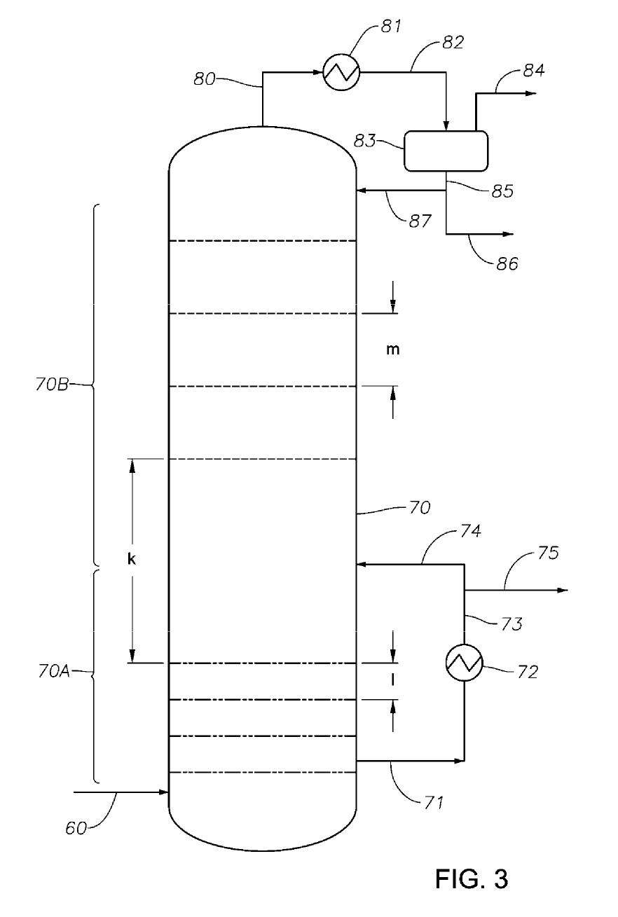

It is observed that when utilizing a conventional fractionator, such as the one illustrated in FIG. 2, increasing the flow rate of the process stream to obtain an increase in the fractionator's C_{f}≧0.33 ft/second (0.101 m/s) overhead volumetric flow rate results in an undesirable increase in the separated naphtha's final boiling point, discoloration of the separated naphtha (the naphtha becomes black), and a decrease in the amount of gas oil that is removed from stage 70 via line 71. Referring now to FIG. 2, the process stream can be conducted to the vicinity of the bottom head of fractionator 70 via conduit 60. Fractionator 70 can be of conventional size and shape, e.g., a substantially cylindrical vessel having inside diameter ≧1.0 m and a length ≧5.0 m. Although this feature is not shown in FIG. 2 or 3, the fractionator vessel can optionally have upper and lower regions (e.g., an upper rectification region and a lower stripping region), with the inside diameter of the upper region being, e.g., in the range of from about 0.85 to about 0.95 times that of the lower region.

Referring again to FIG. 2, the lower region 70A of the conventional fractionator can be a stripping region, with the upper level 70B being a rectification region. The stripping region is utilized for disengaging less-volatile process stream components from a vapor phase in order to concentrate these in a liquid phase. The rectification region is utilized for disengaging more-volatile components from the liquid phase in order to concentrate these in the vapor phase. Generally, region 70A comprises <50.0% of the vessel's internal volume, e.g., in the range of about 20% to about 45%, and region 70B comprises ≧50.0% of the vessel's internal volume, e.g., in the range of about 55% to about 80%.

The stripping region 70A of the conventional fractionator of FIG. 2 can be similar to or substantially the same such as the one described for “diesel oil” separation in European Patent Application No. 2554228 A1, which is incorporated by reference herein in its entirety. For example, the stripping region can contain three, four, or five trays, e.g., a group of four substantially identical, equally-spaced, dual-flow fractionator trays, as indicated by the four horizontal broken lines. Each tray can be spaced apart from its nearest neighbor(s) along the vessel's long axis. The trays can be substantially equally-spaced, e.g., having an inter-tray spacing “i”≧300 mm, e.g., in the range of about 300 mm to about 1220 m, such as in the range of 300 mm to 600 mm, or from about 600 mm to about 1200 mm. In certain embodiments, the inter-tray spacing is in the range of about 300 mm to about 900 mm. The trays can have an open area <0.30, e.g., in the range of from about 0.15 to about 0.30. The trays' perforations are generally arranged in a periodic array having a regular pattern. The perforations are substantially circular, and have a diameter in the range of from about 13 mm (0.51 inches) to about 24 mm (0.95 inches). A conventional rectification region of the type illustrated in FIG. 2 can utilize a second group of equally-spaced dual-flow trays, such as the group disclosed for “gasoline separation” in European Patent Application No. 2554228 A1. The trays of second group of equally-spaced dual-flow fractionator trays are shown as nine horizontal broken lines in region 70B. The first group of trays is spaced apart from the second group of trays by a distance h. Conventional inter-group spacing, as shown in the figures of European Patent Application No. 2554228 A1, is on the order of 2 to 6 times the spacing of the trays within a group. For the first and second tray groups shown in FIG. 2, the inter-group spacing h can be, e.g., in the range of about 1.2 m to about 1.8 meters.

FIG. 2 schematically shows a conventional rectification region, the rectification region being utilized for gasoline separation. One conventional rectification section is disclosed in European Patent Application No. 2554228 A1, which utilizes a group (a second group) of seven to ten trays, e.g., a group of nine substantially identical, equally-spaced, dual-flow fractionator trays. Other references disclose an even greater number of trays in the rectification region, e.g., 20 valve trays or 14 ripple trays™, as disclosed in The Definitive Solution: Gasoline Fractionator Retrofit Techniques, Joseph M. Gondolphe and Casey L. Mueller; Proceedings of the 14th Ethylene Producers' Conference, AIChE, 2002, which is incorporated by reference herein in its entirety. For the conventional rectification region of FIG. 2, the trays have an inter-tray spacing “j”≦about 30 inches (760 mm), e.g., in the range of from about 11.8 inches (300 mm) to about 23.6 inches (600 mm). The trays have an open area <0.30, e.g., in the range of from about 0.15 to about 0.30. The trays' perforations are arranged in a periodic array having a regular pattern. The perforations are substantially circular, and have a diameter in the range of from about 13 mm (0.51 inches) to about 18 mm (0.71 inches). Line 80 is positioned above the second group of trays, e.g., proximate to the fractionator head, to receive the separated vapor phase.

It has been found that utilizing a fractionator, such as the one illustrated in FIG. 3, increasing the flow rate of the process stream to achieve a C_{f}≧0.33 feet/second (0.101 m/s), e.g., C_{f}≧0.107 m/s, such as C_{f}≧0.112 m/s, results in little or no increase in the separated naphtha's final boiling point, little or no discoloration of the separated naphtha (the naphtha does not become black), and little or no decrease in the amount of gas oil that is removed from stage 70 via line 71. In certain embodiments, the invention is practiced in combination with conventional methods for lowering the naphtha's final boiling point, such as changing the steam: hydrocarbon mass ratio in the feed to the pyrolysis furnace's radiant section, but these are not required. Generally, ≧95.0 wt. % of the gas oil, based on the weight of the gas oil, has an atmospheric boiling point that is greater than the separated naphtha's final boiling point. The fractionator of FIG. 3 can be of substantially the same internal size and shape as the conventional fractionator of FIG. 2.

The stripping region 70A of the fractionator of the invention, shown in FIG. 3, can be similar to or substantially the same as that of the conventional stripping region described in the preceding paragraphs, such as the stripping region illustrated in FIG. 2. The tray type, tray number, tray spacing, open area, etc., can be the same as described for the conventional stripping region, e.g., the trays of the stripping region can have an inter-tray spacing “1” that is substantially the same as the inter-tray spacing of the trays of the stripping region of the conventional fractionator “i”. As in the conventional fractionator, the rectification region of the fractionator of FIG. 3 utilizes a second group of equally-spaced dual-flow fractionator trays, as indicated by the four horizontal broken lines in region 70B. The first group of trays is spaced apart from the second group of trays by a distance k. The value of k is not critical, and can be, e.g., in the range of from about 0.9 m to about 2.1 m, such as about 1.2 m to about 1.8 m. Besides the trays of the first and second group, means can be utilized in the fractionator for more evenly distributing the process stream across the fractionator's cross-sectional area. Conventional chimney trays can be utilized to do this, such as one or more chimney trays located below the first tray of the first group. Optionally, gas oil has an initial boiling point ≧288° C., and is conducted away via line 71 at a rate ≧2.0×10³ gallons per minute (7.6×10³ liters/minute), e.g., rate ≧2.5×10³ gallons per minute (9.5×10³ liters/minute). The invention is compatible with conventional fractionator technology, such as that disclosed in European Patent Application No. 2554228 A1, including means utilized for conducting the process stream to the fractionator, means for distributing the process stream in the fractionator, means for circulating quench oil, baffle means in the lower region of the fractionator, means for removing naphtha from the fractionator, reflux means, etc., but the invention is not limited thereto.

The rectification region of the fractionator of FIG. 3 utilizes a group of three, four, or five substantially identical, equally-spaced, dual-flow fractionator trays. In certain embodiments four trays are utilized. Optionally, the trays are Ripple Trays™. The trays have an inter-tray spacing “m” in the range of from about 36 inches (910 mm) to about 50 inches (1270 mm), e.g., about 46 inches to about 50 inches (about 1160 mm to about 1270 mm) The trays have an open area ≧0.25, such as ≧0.26, or ≧0.28, e.g., in the range of from about 0.26 to about 0.30. Optionally, the trays of the first group have an open area that is less than that of the second group's trays, e.g., ≦0.9 times the open area of the trays of the second group. Generally, the trays' perforations are arranged in a periodic array having a regular pattern. The perforations can be substantially circular, and can have, e.g., a diameter in the range of from about 13 mm (0.51 inches) to about 25 mm (1.0 inch), such as 19 mm (0.75 inch) to 25 mm (1.0 inch). The trays can have, e.g., a pitch to diameter ratio in the range of about 1.7 to about 1.8. In the embodiments illustrated in FIG. 3, each tray of the first group is separated from its neighboring tray(s) by substantially the same distance (1), and each tray of the second group is separated from its neighboring tray(s) by substantially the same distance (m). In other embodiments, the distance between each of a group's trays is not the same, e.g., the spacing between a first pair of trays in a group can be ≧5% larger or smaller than the spacing between a second pair of trays in the group, e.g., ≧10% larger or smaller.

Additional separation stages, not shown, can be utilized upstream or downstream of stages 70 and 83, e.g., one or more tar drums, water-quench towers, indirect condensers, etc., such as those described in U.S. Pat. No. 8,083,931, which is incorporated by reference herein in its entirety. For example, stages for removing heat (such as one or more transfer line heat exchangers) and removing tar (such as tar drums) can be located upstream of stage 70 if desired. As shown in FIGS. 1 and 3, an upgraded vapor stream is conducted away from stage 83 via conduit 84, the upgraded vapor stream comprising ≧5.0 wt. %, e.g., 7.5 wt. %, such as ≧10.0 wt. % of the C₂₋₄ olefin present in the process stream, based on the weight of the process stream. Water can be conducted away from stage 83 by one or more conduits (not show). In certain embodiment, e.g., those illustrated in FIG. 1, the upgraded vapor stream is conducted via line 84 to compression stage 90, and then to one or more stages for removing one or more of the desired C₂, C₃, or C₄ olefins.

For example, the compressed vapor is conducted via line 100 to stage 120 for removing at least a portion of any acidic molecules in the compressed vapor, e.g., H₂S and/or CO₂. After removal of these acidic molecules in stage 120, and an upgraded olefin-containing stream is conducted away from stage 120 via line 200. Conventional methods for removing acid gases can be utilized in stage 120, although the invention is not limited thereto, e.g., amine and/or caustic contactors. For example, at least a portion of the compressed vapor's acidic gases, e.g., H₂S and/or CO₂ can be transferred to an aqueous amine mixture in stage 120, with these molecules (or ionized fragments thereof) being conducted away as components of the rich aqueous amine mixture via line 130.

In certain embodiments, at least a portion of any water in the upgraded olefin-containing stream can be removed in stage 210, which can utilize e.g., conventional drying technology, though the invention is not limited thereto. A dried olefin-containing stream is conducted away from stage 210 via line 220 and is then exposed to a temperature of, e.g., 100.0° C. in stage 230 to produce a cooled stream. The cooled stream is conducted via line 240 to separation stage 250, where a stream comprising primarily a mixture of methane and molecular hydrogen is conducted away via line 270. If desired, molecular hydrogen (line 290) and/or methane (line 300) can be separated from the mixture, utilizing, e.g., one or more cold boxes 280.

A demethanized stream can be conducted away from separation stage 250 via line 260 to separation stage 310 for separating at least a portion of any C₂ hydrocarbon from the demethanizer stream. The C₂ hydrocarbon (generally a mixture of ethane, ethylene, and acetylene) can be conducted away from stage 310 via line 320 to acetylene conversion stage 330. Stage 330 utilizes molecular hydrogen 340 (obtained, e.g., from line 290) and a catalytically effective amount of at least one acetylene conversion catalyst operating under acetylene conversion conditions to convert at least a portion of the acetylene to ethylene. An upgraded C₂ hydrocarbon mixture is conducted away from stage 330 via line 350 to separation stage 360 (e.g., a splitter) for separating ethylene (conducted away via line 370) from ethane (conducted away via line 380). The ethylene can be conducted away for storage or further processing, e.g., polymerization.

C₃₊ hydrocarbon is conducted away from stage 310 via line 390, e.g., for separating propylene and/or C₄ olefin generally (including isoolefin), which can be conducted away for storage and/or further processing, e.g., polymerization.

All patents, test procedures, and other documents cited herein, including priority documents, are fully incorporated by reference to the extent such disclosure is not inconsistent and for all jurisdictions in which such incorporation is permitted.

While the illustrative forms disclosed herein have been described with particularity, it will be understood that various other modifications will be apparent to and can be readily made by those skilled in the art without departing from the spirit and scope of the disclosure. Accordingly, it is not intended that the scope of the claims appended hereto be limited to the examples and descriptions set forth herein but rather that the claims be construed as encompassing all the features of patentable novelty which reside herein, including all features which would be treated as equivalents thereof by those skilled in the art to which this disclosure pertains.

When numerical lower limits and numerical upper limits are listed herein, ranges from any lower limit to any upper limit are contemplated. The term “comprising” is synonymous with the term “including”. Likewise whenever a composition, an element or a group of components is preceded with the transitional phrase “comprising”, it is understood that we also contemplate the same composition or group of components with transitional phrases “consisting essentially of”, “consisting of”, “selected from the group of consisting of”, or “is” preceding the recitation of the composition, component, or components, and vice versa.

What is claimed is:

1. A fractionator, comprising:
(a) an elongated fractionator vessel having an internal volume ≧about 28.3 m³ and a C_{f}≧0.33;
(b) a first group of three, four, or five perforated trays in the fractionator, each tray of the first group being (i) a dual-flow tray having an open area in the range of 0.15 to 0.30 and (ii) adapted to substantially prevent fluid flow between the tray's perimeter and the fractionator vessel's inner surface;
(c) a second group of three, four, or five perforated trays in the fractionator, the second group being spaced apart from the first group along the fractionator vessel's long axis, each tray of the second group being (i) a dual-flow tray having an open area ≧0.25 and (ii) adapted to substantially prevent fluid flow between the tray's perimeter and the fractionator vessel's inner surface;
(d) a first conduit for conducting to the fractionator vessel a first mixture comprising naphtha, quench fluid, and ≧10.0 wt. % C₂₋₄ olefin based on the weight of the first mixture;
(e) a second conduit for conducting away a second mixture from the fractionator vessel, the second mixture comprising naphtha and C₂₋₄ olefin, the second mixture being separated from the first mixture in the fractionator vessel, the naphtha having a final boiling point ≦288° C.; and
(f) a third conduit for conducting away from the fractionator vessel a gas oil that is separated from the first mixture in the fractionator vessel, the gas oil having an initial boiling point that is greater than or equal to the naphtha's final boiling point.

2. The fractionator of claim 1, wherein the fractionator's internal volume is ≧142 m³.

3. The fractionator of claim 1, wherein the fractionator vessel has a first region, the first region being proximate to the first group of trays and having (i) a substantially circular internal cross section and (ii) an inside diameter ≧3.05 m.

4. The fractionator of claim 3, wherein the fractionator vessel has a second region, the second region being proximate to the second group of trays and having a (i) substantially circular internal cross section and (ii) an inside diameter ≧3.05 m, wherein the first region has an internal cross sectional area that is greater than that of the second region.

5. The fractionator of claim 1, wherein each tray of the second group is spaced apart from its nearest-neighbor tray(s) by an average spacing in the range of about 910 mm to about 1270 mm.

6. The fractionator of claim 1, wherein each tray of the first group is spaced apart from its nearest-neighbor tray(s) by an average spacing in the range of about 600 mm to about 1200 mm.

7. The fractionator of claim 1, wherein each tray of the second group is a corrugated sieve tray having no downcomers, and wherein the perforations of each tray of the second group (i) are substantially circular, (ii) have a diameter in the range of about 13 mm to about 25 mm, and (iii) have an open area ≧0.25.

8. The fractionator of claim 1, wherein the perforations of each tray of the first group (i) are substantially circular, (ii) have a diameter in the range of about 13 mm to about 25.4 mm, and (iii) have an open area ≦0.30.

9. The fractionator of claim 1, wherein the first group contains 4 trays and the second group contains 4 trays.

10. The fractionator of claim 1, wherein the open area of each tray of the second group is ≧0.28.

11. A vapor-liquid separation system, comprising:
(a) first and second vessels, the first vessel being elongated and having an internal volume ≧about 28.3 m³ and a C_{f}≧0.101 m/s;
(b) a first region of the first vessel, the first region having three, four, or five perforated trays, each tray being (i) a dual-flow tray that is spaced apart from its nearest-neighbor tray(s) along the first vessel's long axis and (ii) positioned to substantially prevent fluid flow between the tray's perimeter and the first vessel's internal surface, wherein each tray has an open area in the range of about 0.15 to about 0.30 and the tray's perforations have a size in the range of from about 13 mm to about 25 mm;
(c) a second region of the first vessel, the second region being substantially non-overlapping with the first region and having three, four, or five perforated trays, each tray being (i) a dual-flow tray that is spaced apart from its nearest-neighbor tray(s) along the first vessel's long axis and (ii) positioned to substantially prevent fluid flow between the tray's perimeter and the first vessel's internal surface, wherein each tray has an open area ≧0.25 and the tray's perforations have a size in the range of from about 13 mm to 25 mm;
(d) a first conduit for transferring to the first vessel's first region a first mixture comprising quench fluid, naphtha, and ≧10.0 wt. % C₂₋₄ olefin based on the weight of the first mixture;
(e) a second conduit for transferring a second mixture from the first vessel's second region to the second vessel, the second mixture comprising C₂₋₄ olefin that is separated from the first mixture in the first vessel;
(f) a third conduit for conducting away from the first vessel's first region a gas oil that is separated from the first mixture in the first vessel, the gas oil having an initial boiling point ≧about 221° C.;
(g) a fourth conduit for conducting a liquid naphtha away from the second vessel, wherein the liquid naphtha (i) is disengaged from the second mixture in the second vessel and (ii) has a final boiling point ≦288° C.;
(h) a fifth conduit connected to the fourth conduit for transferring a first portion of the liquid naphtha from the fourth conduit to the to the second region of the first vessel;
(i) a sixth conduit connected to the fourth conduit for conducting away from the fourth conduit a second portion of the liquid naphtha; and
(j) a seventh conduit connected to the second vessel for conducting away a vapor from the second vessel, wherein the vapor (i) comprises C₂₋₄ olefin and (ii) is disengaged from the second mixture in the second vessel.

12. The system of claim 11, wherein the gas oil has an initial boiling point ≧288° C.

13. The system of claim 11, wherein the fractionator's C_{f} is ≧0.107 m/s.

14. The system of claim 11, wherein the fractionator's C_{f} is ≧0.112 m/s.

15. The system of claim 11, wherein the gas oil is conducted away from the first vessel at a rate ≧7.6×10³ liters per minute.

16. The system of claim 11, wherein the first vessel's internal volume ≧142 m³.

17. The system of any of claims 11-16 wherein the first region has a substantially circular internal cross section and has an inside diameter ≧3.05 m, the first region having an internal cross sectional area that is substantially the same as or greater than that of the second region.

18. The system of claim 11, wherein (i) each tray of the second region is spaced apart from its nearest-neighbor tray(s) by an average spacing in the range of about 910 mm to about 1270 mm and (ii) each tray of the first region is spaced apart from its nearest-neighbor tray(s) by an average spacing in the range of about 600 mm to about 1200 mm.

19. The system of claim 11, wherein the open area of each tray of the second region is ≧0.28.

20. The system of claim 11, wherein the second vessel is a flash drum.

21. A hydrocarbon conversion process, comprising:
(a) providing a feedstream, the feedstream comprising ≧75.0 wt. % hydrocarbon based on the weight of the feedstream;
(b) exposing the feedstream to a temperature ≧400° C. under pyrolysis conditions to produce an effluent comprising ≧5.0 wt. % of C₂₋₄ olefin based on the weight of the feedstream, wherein the pyrolysis is conducted at a feedstream mass flow rate ≧113×10³ kg per hour and a feedstream:water mass ratio in the range of from about 2.0 to 2.5;
(c) combining the effluent with a quench fluid to produce a first mixture;
(d) conducting the first mixture to a first separation zone having a C_{f}≧0.101 m/s, the first separation zone comprising substantially non-overlapping first and second regions, wherein (i) the first region contains three, four, or five dual-flow trays and (ii) the second region contains three, four, or five dual-flow trays;
(e) separating a second mixture from the first mixture in the first separation zone, and transferring the second mixture from the first separation zone to a second separation zone, the second mixture comprising C₂₋₄ olefin that is separated from the first mixture in the first separation zone;
(f) separating a gas oil from the first mixture in the first separation zone, the gas oil having an initial boiling point ≧about 221° C.; and
(g) separating a liquid naphtha from the second mixture in the second separation zone, the liquid naphtha having a final boiling point ≦288° C.

22. The process of claim 21, further comprising
(a) dividing the liquid naphtha into first and second portions, and transferring the first portion of the liquid naphtha to the second region of the first separation zone and conducting away the second portion of the liquid naphtha;
(b) dividing the separated gas oil into first, second, and third portions, wherein (i) the quench fluid comprises ≧90.0 wt. % of the first gas oil portion, based on the weight of the quench fluid, (ii) the second portion of the separated gas oil is conducted away from the process, and (iii) a third portion of the separated gas oil is returned to the first separation zone; and
(c) conducting away a vapor from the second vessel, wherein the vapor (i) comprises C₂₋₄ olefin and (ii) is disengaged from the second mixture in the second vessel's disengagement zone.

23. The process of claim 21, wherein the second region of the first separation zone contains four dual-flow trays, each tray (i) being spaced apart from its nearest-neighbor tray(s) by an average spacing in the range of about 910 mm to about 1270 mm and (ii) having an open area ≧0.28.

24. The process of claim 21, wherein C_{f} is ≧0.112 m/s and the gas oil is separated in the first separation zone at a rate ≧7.6×10³ liters/minute.

25. The process of claim 22, wherein the first portion of the liquid naphtha has a luminance ≧5%.
